(19) United States
(12) Patent Application Publication
(10) Pub. No.: US 2025/0276106 A1
(43) Pub. Date: Sep. 4, 2025

(54) ECOFRIENDLY SKIN SAFE ABSORBENT

(71) Applicant: TEXAS STATE UNIVERSITY, San Marcos, TX (US)

(72) Inventors: Sujata Mandal, San Marcos, TX (US); Sangchul Hwang, Austin, TX (US)

(73) Assignee: TEXAS STATE UNIVERSITY, San Marcos, TX (US)

(21) Appl. No.: 19/067,951

(22) Filed: Mar. 2, 2025

Related U.S. Application Data
(60) Provisional application No. 63/560,055, filed on Mar. 1, 2024.

(51) Int. Cl. A61L 15/28; A61L 15/60; C08L 5/00; C08L 5/04; C08L 77/04

(52) U.S. Cl. CPC A61L 15/28; A61L 15/60; C08L 5/00; C08L 5/04; C08L 77/04; A61L 2300/23; A61L 2300/252

(57) ABSTRACT

Embodiments of the present disclosure pertain to a hydrogel that includes one or more galactomannans and one or more biodegradable polymers. Additional embodiments of the present disclosure pertain to absorptive hygiene products (AHPs) that include the hydrogels of the present disclosure. The AHP may include, without limitation, diapers, tampons, sanitary pads, or combinations thereof. Further embodiments of the present disclosure pertain to methods of forming the hydrogels of the present disclosure. Such methods include associating one or more galactomannans with one or more biodegradable polymers. The methods of the present disclosure may also include a step of incorporating the hydrogel as a component of an AHP.

### CROSS-REFERENCE TO RELATED APPLICATIONS

This application claims priority to U.S. Provisional Patent Application No. 63/560,055, filed on Mar. 1, 2024. The entirety of the aforementioned application is incorporated herein by reference.

### BACKGROUND

A need exists to develop biodegradable polymers for use in numerous products, such as hygiene products. Numerous embodiments of the present disclosure aim to address the aforementioned need.

### SUMMARY

In some embodiments, the present disclosure pertains to a hydrogel that includes: one or more galactomannans; and one or more biodegradable polymers. The hydrogels of the present disclosure can have various advantageous properties. For instance, in some embodiments, the hydrogels of the present disclosure are biodegradable. In some embodiments, the hydrogels of the present disclosure are operable to retain liquids. In some embodiments, the liquids include urea.

Additional embodiments of the present disclosure pertain to absorptive hygiene products (AHPs) that include the hydrogels of the present disclosure. In some embodiments, the AHP includes, without limitation, diapers, tampons, sanitary pads, or combinations thereof. In some embodiments, the AHP includes diapers.

Further embodiments of the present disclosure pertain to methods of forming the hydrogels of the present disclosure. Such methods include associating one or more galactomannans with one or more biodegradable polymers. In some embodiments, the methods of the present disclosure also include a step of incorporating the hydrogel as a component of an AHP.

### FIGURES

FIG. 1A illustrates the synthesis of sustainable superabsorbent polymers (SAPs) from guar gum and alginate (GG-AA-SAP) for absorbent hygiene products (AHPs).

FIG. 1B shows an image of the fabricated GG-AA-SAP.

FIG. 2A shows a tea bag method absorbency study for GG-AA-SAP.

FIG. 2B shows the maximum swelling of GG-AA-SAP. W2 is wet GG-AA-SAP and W1 is dry GG-AA-SAP.

FIG. 3A shows the addition of artificial urine to GG-AA-SAP and in store diaper (treated as control).

FIG. 3B shows a commercial AHP (baby diaper) treated with artificial urine.

FIG. 3C shows a GG-AA-SAP treated with artificial urine.

FIGS. 4A-4B compare the biodegradability of GG-AA-SAP (FIG. 4A) and baby diaper (FIG. 4B) in 5 g of soil after 72 hours.

FIGS. 5A-5B compare the biodegradability of GG-AA-SAP (FIG. 5A) and baby diaper (FIG. 5B) in 5 g of soil after 5 days.

FIGS. 6A-6B provide data regarding the weight bearing ability of fabricated GG-AA-SAP.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Superabsorbent polymers (SAPs) are a class of high-performance synthetic polymers that are distinguished by their outstanding water absorption capacity. SAPs derived from petroleum have been widely used as the primary component of absorptive hygiene products (AHPs).

The diaper and hygiene product industry caters to a significant customer base that includes infants (e.g., use of diapers), the elderly (e.g., addressing incontinence), and adults with certain medical conditions. The diaper and hygiene industry has a market share of over 92%.

The most common SAPs currently employed are disposable and have a petrochemical base that includes poly(acrylic acid) salts and poly(acrylamide) since their monomers are developed from nonrenewable petrochemical sources. Commercial SAPs are usually obtained by free-radical polymerization of vinylic monomers with multifunctional cross-linkers. Some of the critical downsize of synthetic polymers are non-recyclability, high market price, energy-consuming synthesis processes, toxicity of trace initiators and unreacted monomers, and environmental concerns.

Driven by ease of use of ‘in store’ SAPs, one major drawback of these one-time use disposable diapers and AHPs is that most of them invariably end up in landfills. It is estimated that the decomposition time of disposable diapers and AHPs is about half of a millennium, owing to the presence of synthetic components.

A disposable diaper typically contains several polymers, such as polyethylene, polypropylene, polyesters and polyacrylates. Of these constituents, the most recent addition to the disposable diaper is the superabsorbent polymer (SAP) sodium polyacrylate, which is non-biodegradable, and which is the primary agent for water absorption and retention located in the core of the commodity product.

Most personal care products are made from superabsorbent materials, which are mainly composed of sodium polyacrylate, have a high-water absorbing quality, and can absorb 200 to 300 times their weight. Unlike natural polymers that are broken down by microorganisms to get smaller molecules, the C—C single bond of polymers found in diapers cannot be easily degraded by most microorganisms.

Most of the diapers are not easily biodegradable and have an estimated period of up to 500 years to decompose. Disposable diapers thus have bad effects on the environment, as they are a form of solid waste.

In fact, disposable diapers are the third largest contributor to municipal solid waste in the United States of America, accounting for 1.5% to 4% of total waste. Apart from the solid waste problem, chemicals released by decomposing solid wastes can leak from dumping sites and landfills to groundwater. They can contaminate water wells, soil, and nearby water streams. Additionally, the chemicals can release some harmful gases, drainage clogging, and water contamination. In view of the aforementioned problems, a need exists to improve the biodegradability of AHPs.

As such, a need exists to develop new biodegradable SAP substitutes for synthetic polymers, such as sodium polyacrylate. A need also exists for the development of products, such as hygiene products, that utilize such SAP substitutes. Numerous embodiments of the present disclosure aim to address the aforementioned needs.

In some embodiments, the present disclosure pertains to a hydrogel that includes: one or more galactomannans; and one or more biodegradable polymers. Additional embodiments of the present disclosure pertain to absorptive hygiene products (AHPs) that include the hydrogels of the present disclosure. Further embodiments of the present disclosure pertain to methods of forming the hydrogels of the present disclosure by associating one or more galactomannans with one or more biodegradable polymers. As set forth in more detail herein, the hydrogels, AHPs and hydrogel formation methods of the present disclosure can have numerous embodiments.

### Galactomannans

The hydrogels and AHPs of the present disclosure can include various galactomannans. Additionally, the hydrogel formation methods of the present disclosure may utilize various galactomannans. For instance, in some embodiments, the galactomannans include, without limitation, natural gums, guar gum, fenugreek gum, xanthan gum, locust bean gum, Arabic gum, tara gum, tragacanth gum, cassia gum, gum karaya, gum ghatti, gellan gum, kondagogu gum, gum tamarind, or combinations thereof.

In some embodiments, the galactomannans facilitate the formation of gels, stabilization of emulsions, and modification of the texture of hydrogels. In some embodiments, the galactomannans include guar gum. In some embodiments, the galactomannans include natural gums. Natural gums are obtained from renewable sources, such as microbial, marine, or plant sources, which encompass many biospheres. Natural gums have various advantages, such as availability, low cost, structural diversity, renewability, biocompatibility, biodegradability, non-toxicity, and the ability to undergo chemical modifications.

### Biodegradable Polymers

Biodegradable polymers refer to polymers that are capable of breaking down completely through the action of microorganisms. For instance, in some embodiments, the biodegradable polymers may break down into natural byproducts, such as gases (e.g., CO₂, N₂), water, biomass, and inorganic salts.

The hydrogels and AHPs of the present disclosure can include various biodegradable polymers. Additionally, the hydrogel formation methods of the present disclosure may utilize various biodegradable polymers. For instance, in some embodiments, the biodegradable polymers include, without limitation, agro-polymers, polysaccharides, proteins, poly amino acids, biopolymers, bio-polyesters, or combinations thereof.

In some embodiments, the biodegradable polymers include poly amino acids. In some embodiments, the poly amino acids include poly glutamic acid (PGA). PGA is a water-soluble and biodegradable linear molecule with a relatively high molecular weight (e.g., 100,000 to 1,000,000 g/mol). Naturally, PGA occurs in organisms by the condensation of glutamate and ammonia and plays an essential role in the metabolism of nitrogen. In humans, PGA facilitates natural processes in the intestines, regulates acid-base balance in the kidneys, activates immune cells, and feeds cancerous cells.

In some embodiments, the biodegradable polymers include one or more polysaccharides. In some embodiments, the polysaccharides include, without limitation, starch, glycogen, galactogen, arabinoxylans, cellulose, chitin, chitosan, pectin, starch, alginic acid, or combinations thereof. In some embodiments, the polysaccharides include alginic acid. In some embodiments, the polysaccharides include chitosan.

In some embodiments, the biodegradable polymers include one or more biopolymers. Biopolymers are naturally occurring polymers that are derived from renewable sources, such as plants, animals, and microorganisms. In some embodiments, biopolymers include, without limitation, polysaccharides, proteins, polylactic acid (PLA), bio-polyesters (i.e., polyesters derived from biological sources), polybutylene succinate (PBS), or combinations thereof.

### Arrangements of Galactomannans and Biodegradable Polymers

The hydrogels and AHPs of the present disclosure can include various arrangements of galactomannans and biodegradable polymers. For instance, in some embodiments, the galactomannans and the biodegradable polymers are covalently cross-linked to one another. Covalent cross-linking refers to the formation of strong, permanent bonds between the polymer chains, often through chemical reactions involving functional groups on the polymer molecules. This process can increase a biodegradable polymer's mechanical strength, stability, and resistance to degradation in certain conditions, while still maintaining its biodegradable properties.

In some embodiments, the biodegradable polymers include poly glutamic acid (PGA) and the galactomannans include guar gum. In some embodiments, the PGA and the guar gum are covalently cross-linked to one another. In some embodiments, the biodegradable polymers include alginic acid and the galactomannans include guar gum. In some embodiments, the alginic acid and the guar gum are covalently cross-linked to one another.

### Hydrogel Properties

The hydrogels of the present disclosure can have various advantageous properties. For instance, in some embodiments, the hydrogels of the present disclosure are biodegradable. In some embodiments, the biodegradable hydrogels may break down into natural byproducts, such as gases (e.g., CO₂, N₂), water, biomass, and inorganic salts.

In some embodiments, the hydrogels of the present disclosure are operable to retain liquids. In some embodiments, the liquids include urea.

In some embodiments, the hydrogels of the present disclosure are operable to retain liquids at more than 100 times the hydrogel's weight. In some embodiments, hydrogels of the present disclosure are operable to retain liquids at more than 300 times the hydrogel's weight. In some embodiments, hydrogels of the present disclosure are operable to retain liquids at more than 500 times the hydrogel's weight. In some embodiments, hydrogels of the present disclosure are operable to retain liquids at more than 700 times the hydrogel's weight.

### Absorptive Hygiene Products (AHPs)

In some embodiments, the hydrogels of the present disclosure are a component of an absorptive hygiene product (AHP). Additional embodiments of the present disclosure pertain to AHPs that include the hydrogels of the present disclosure. In some embodiments, the AHP includes, without limitation, diapers, tampons, sanitary pads, or combinations thereof.

In some embodiments, the AHP includes diapers. Diapers generally consists of multiple layers and materials that together provide comfort, leak protection, and absorbency. A proposed structure for a diaper as an AHP includes: (a) a top sheet (e.g., a cover layer of non-woven soft fabric); (b) an absorbent core (e.g., a layer that can absorb large quantities of liquid, expanding to retain moisture and prevent leakage); (c) an acquisition layer (e.g., a layer that helps to distribute the liquid evenly across the absorbent core, thereby preventing localized saturation); and (c) an outer layer (e.g., the external part of the diaper that provides a waterproof barrier to prevent leakage). In some embodiments, the hydrogels of the present disclosure include the absorbent core. In some embodiments, the hydrogels of the present disclosure provide an outer shape and structure of the diaper.

### Methods of Making Hydrogels

Additional embodiments of the present disclosure pertain to methods of forming the hydrogels of the present disclosure. Such methods generally include associating one or more galactomannans with one or more biodegradable polymers.

In some embodiments, galactomannans and biodegradable polymers may be associated with one another by mixing the galactomannans with the biodegradable polymers to form a mixture. In some embodiments, the association further includes heating the mixture. In some embodiments, the association includes cross-linking the galactomannans with the biodegradable polymers.

In some embodiments, the methods of the present disclosure also include a step of incorporating the hydrogel as a component of an absorptive hygiene product (AHP). In some embodiments, the AHP includes, without limitation, diapers, tampons, sanitary pads, or combinations thereof. In some embodiments, the AHP includes diapers.

### Advantages and Applications

The hydrogels of the present disclosure can provide numerous advantages. For instance, in some embodiments, the hydrogels of the present disclosure provide safe alternatives to petroleum-based synthetic polymers, as the hydrogels of the present disclosure are developed from natural polymers, have minimum environmental impact, and are more sustainable. Additionally, the hydrogels of the present disclosure are nontoxic, non-allergenic and safe to use. On the other hand, commercial hygiene products are petrochemical-originated, exhibit toxicity, high market price, and pose environmental and public health concerns.

Similarly, the methods of forming the hydrogels of the present disclosure provide numerous advantages. For instance, in some embodiments, the hydrogel formation methods of the present disclosure are facile and ecofriendly. Moreover, the raw materials for the hydrogels of the present disclosure are abundant, benign, and inexpensive.

As such, the hydrogels of the present disclosure can have numerous applications. For instance, in some embodiments, the hydrogels of the present disclosure can be utilized as components of absorptive hygiene products (AHP), such as diapers. In some embodiments, the AHPs are biodegradable. Such biodegradability is advantageous because each baby wears at least 6-8 diapers per day, which are then disposed of in landfills, where they will take 100-500 years to decompose. Additionally, the most alarming constituent of a diaper is sodium polyacrylate, which absorbs 100 times its weight but causes allergy, irritation, fever, infections, and vomiting. These products are even harder to degrade and thereby pollute the ecosystem and release chlorofluorocarbon (CFC), which depletes the ozone layer (protective layer for UV wave). In fact, diapers rank third on the list of materials dumped in landfills.

Moreover, non-biodegradable AHPs are composed of harmful chemicals-dioxins, which can cause an array of health problems, including developmental delays, damaged immunity, hormone interference, skin diseases, and certain types of cancer. Additionally, non-biodegradable AHPs buildup has the potential to disturb natural ecosystems and alter the balance of the organisms, thereby endangering aquatic, marine, and human life. As such, the application of the hydrogels of the present disclosure as biodegradable AHPs can diminish landfill, reduce ozone depletion, reduce soil and ground water pollution, and prevent health issues.

### ADDITIONAL EMBODIMENTS

Reference will now be made to more specific embodiments of the present disclosure and experimental results that provide support for such embodiments. However, Applicant notes that the disclosure herein is for illustrative purposes only and is not intended to limit the scope of the claimed subject matter in any way.

### Example 1. Development of Biodegradable Superabsorbent Polymers (SAPs) and their Use in Diapers

The primary objective of this Example is to develop a biodegradable superabsorbent polymer (SAP) by crosslinking natural gum-based polysaccharides (e.g., guar gum) and renewable biopolymers (e.g., alginic acid). Applicant has introduced several performance metrics to quantitatively compare the potential alternatives: liquid (water and artificial control-urine) absorbance or swelling, and degree of biodegradability.

### Example 1.1. Preparation of Guar Gum (GG)

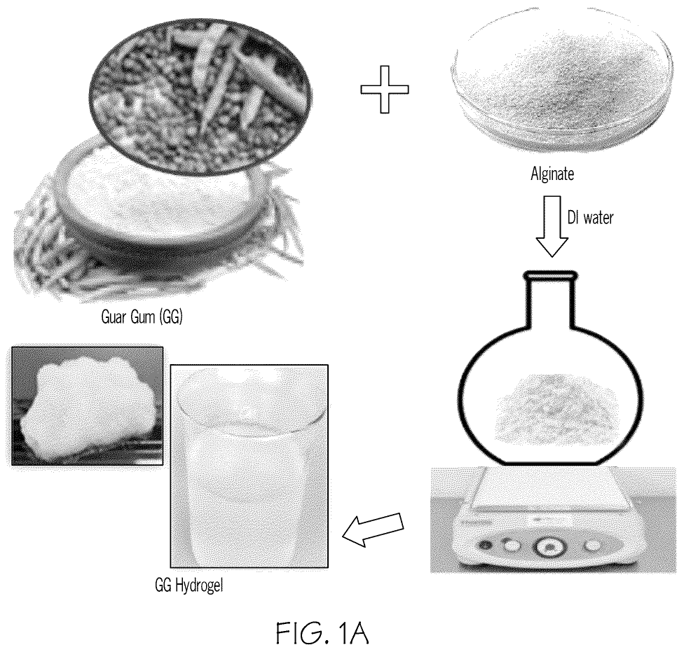

In a round-bottom flask equipped with a mechanical stirrer, 1±0.01 g of guar gum (GG) was dispersed in 50 mL of deionized water and kept under stirring overnight. Next, the solution was sonicated for ˜30 minutes to remove air bubbles until a clear colloidal solution was obtained (FIG. 1A). To the solution, 2 mL of sodium hydroxide was added and stirred for 10 minutes followed by 1 mL of acetic acid. The mixture was centrifuged. The fabricated GG and alginic acid treated hydrogel (FIG. 1B) is termed GG-AA.

### Example 1.2. Preparation of Alginic Acid (AA)-Lyophilization

About 2 g of Alginic acid was dissolved in 100 ml of water and the pH was adjusted to pH 7±0.05 by using sodium hydroxide or hydrochloric acid. To store the acid for further use, the acid was frozen and then vacuum dried, which removed water by sublimation.

### Example 1.3. Preparation of GG-AA SAP

Applicant mixed the alginic acid solution with GG solution prepared earlier while stirring for nearly 1 hour. The mixture was oven dried at 30-40° C.

### Example 1.4. Swelling Properties of the GC-AA SAPs

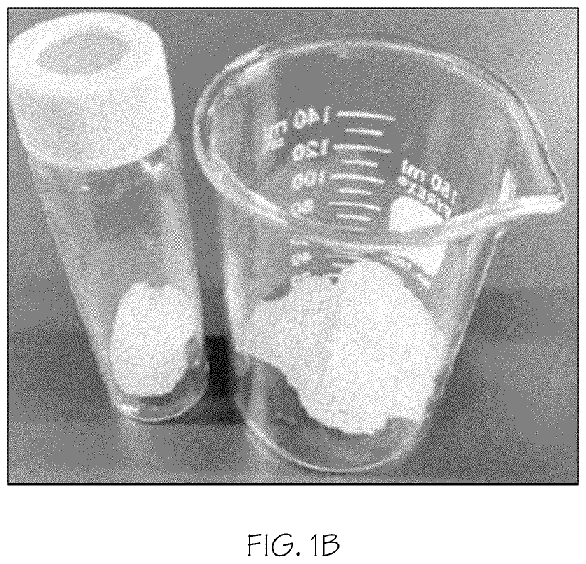

The swelling capacity of the GC-AA SAPs was calculated by a teabag method (FIG. 2A). This method is convenient and the fastest way to examine the absorbency of the absorbent sample (here GG-AA SAP). It is calculated to the amount (g) of liquid absorbed per gram of composition. The tea bag containing dry SAP (0.5 g±0.01) was dissolved in water for 25-30 minutes and then allowed to stand for 10 minutes to release excess water. The maximum absorbency of fabricated GG SAP is also exhibited in FIG. 2B.

The degree of swelling is a quantifiable indication of the polymer's ability to absorb liquid. Applicant can define the degree of swelling as Equation 1.

$$S = \frac{{w}_{2} - {w}_{1}}{{w}_{1}} \qquad Equation 1$$

In equation 1, w₁ is the weight of the GG-AA SAP (before swelling) and w₂ is the weight of the GG-AA SAP (after swelling).

### Example 1.5. Application of GC-AA SAPs as Absorbent Hygiene Products

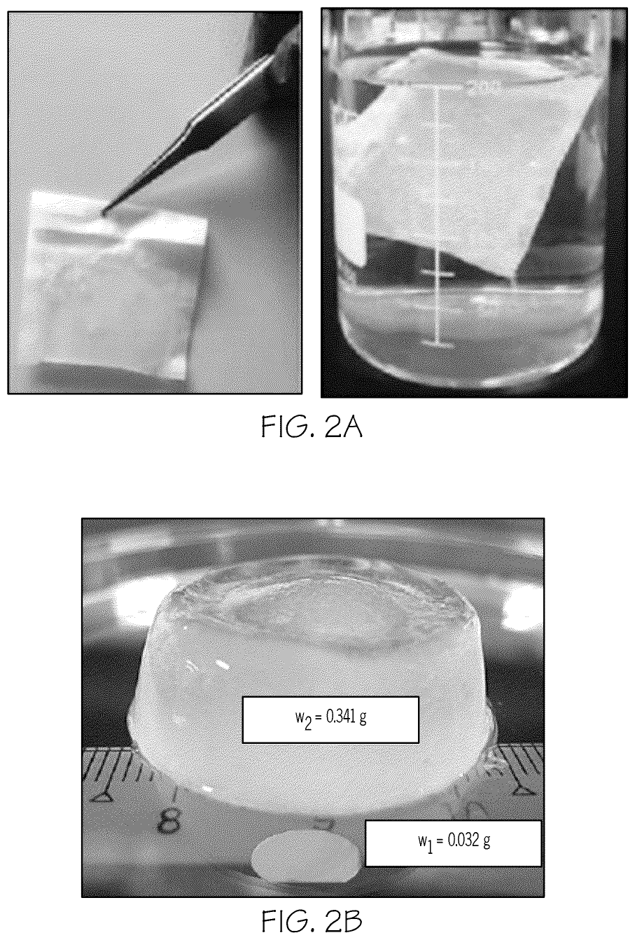

To investigate the efficacy of the prepared GG SAP in terms of urea/urine retention capacity (0.02 g), GG SAP was treated with 1 ml of artificial urine, as shown in FIG. 3A. To compare the efficacy of the fabricated SAP, Applicant used 0.02 g of the ‘in store’ baby diapers. FIGS. 3B and 3C show the images of commercial AHP-baby diaper after 15 minutes of adding 1 ml of urea/artificial urine. It is evident from the above images that the diaper was unable to soak 1 ml of urine as some urea was still left on the weighing boat. On the other hand, two test samples could soak 1 ml of urine very easily.

### Example 1.6. Biodegradability Tests of the GC-AA SAPs

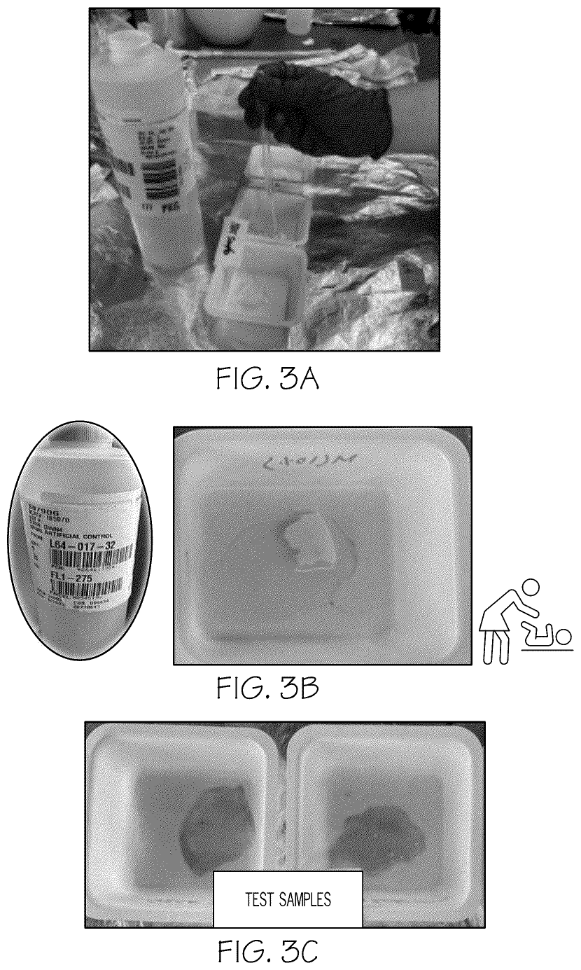

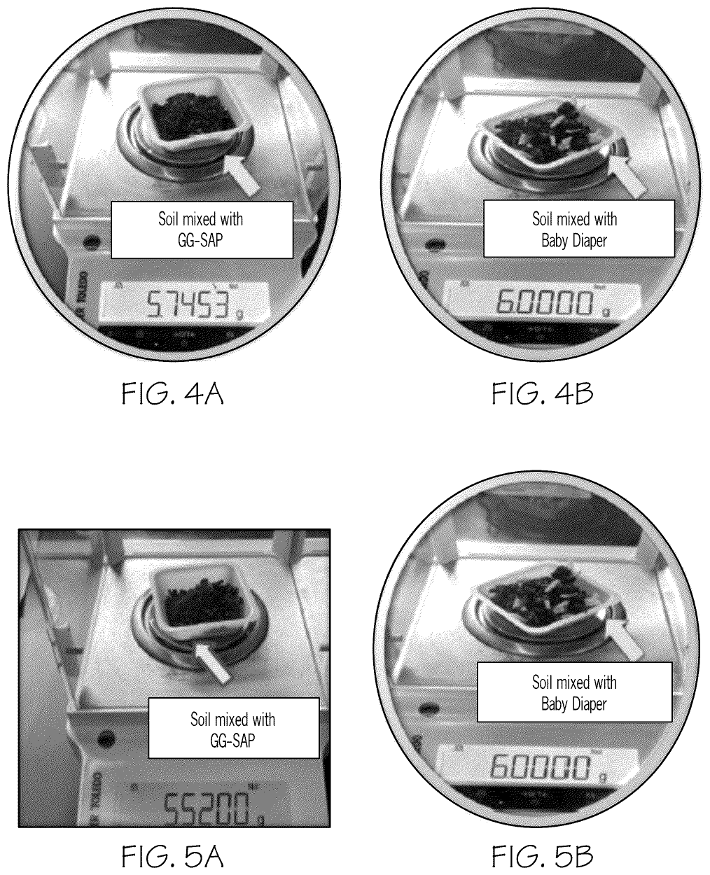

To confirm that GG-AA SAP is ecofriendly, biodegradable, and causes no land fill, Applicant conducted biodegradability tests of the fabricated SAP. About 5 g of dry soil was mixed with 1 g of synthesized SAP (GG-AA) and 1 g of baby diaper. The weight of the soil was measured after 72 hours. As shown in FIGS. 4A-4B and 5A-5B, it is evident that there is significant weight loss of soil mixed with GG-SAP.

### Example 1.7. The Weight Bearing Ability of GC-AA-SAPs

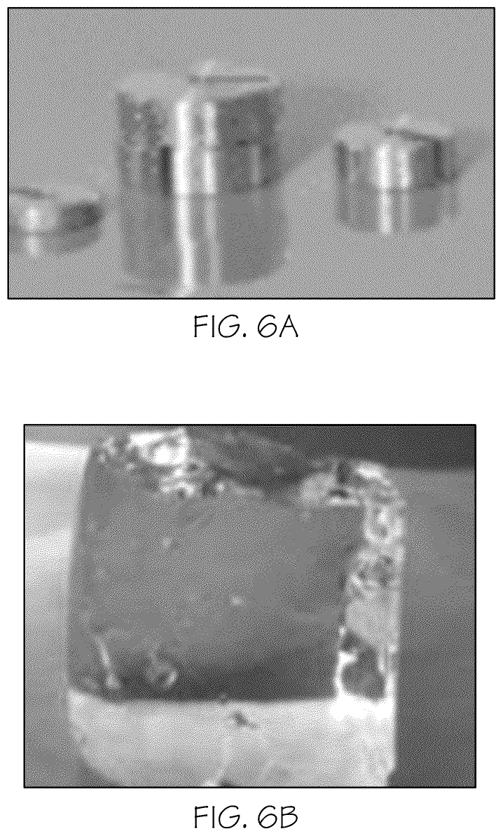

The GC-AA-SPAs were tested for their weight bearing ability. The material was cut into a small cube (2.5 cm×3.0 cm×2.5 cm). The weight was added on the top of the hydrogel, starting from 50 mg to 55 g with an increment of 25 mg. As shown in FIGS. 6A-6B, the material withstood the weight up to 50 g and started compression at 51 g. Cracks were observed at 52 g, and the material yielded at 55 g and collapsed. Therefore, the material had a weight bearing ability of 2.67 g/cm³ (i.e., 50 g per 18.75 cm³).

### Example 1.8. Summary

In addition to its capacity to absorb and retain liquids (water), the developed GC-AA-SAP exhibited optimal affinity with urea and urine. In addition, GC-AA-SAP revealed enhanced biodegradability.

The developed GC-AA-SAP provides numerous advantages. In particular, the developed GC-AA-SAP is a safe alternative to petroleum-based synthetic polymers, as it is developed from natural polymers, has minimum environmental impact, and would be more sustainable in the long term. On the other hand, commercial hygiene products are petrochemical-originated, exhibit toxicity, high market price, and pose environmental and public health concerns. Moreover, AHPs are composed of harmful chemicals-dioxins, which can cause an array of health problems, including developmental delays, damaged immunity, hormone interference, skin diseases, and certain types of cancer.

Additionally, the synthesis method for GC-AA-SAP is facile and ecofriendly. Moreover, the raw materials for GA-AA-SAP are abundant, benign, and inexpensive. Furthermore, the GG-AA-SAP is nontoxic, non-allergenic and safe to use.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A hydrogel comprising:
one or more galactomannans; and
one or more biodegradable polymers.

2. The hydrogel of claim 1, wherein the one or more galactomannans and the one or more biodegradable polymers are covalently cross-linked to one another.

3. The hydrogel of claim 1, wherein the galactomannans are selected from the group consisting of natural gums, guar gum, fenugreek gum, xanthan gum, locust bean gum, Arabic gum, tara gum, tragacanth gum, cassia gum, gum karaya, gum ghatti, gellan gum, kondagogu gum, gum tamarind, or combinations thereof.

4. The hydrogel of claim 1, wherein the galactomannans comprise guar gum.

5. The hydrogel of claim 1, wherein the biodegradable polymers are selected from the group consisting of agro-polymers, polysaccharides, proteins, poly amino acids, biopolymers, bio-polyesters, or combinations thereof.

6. The hydrogel of claim 1, wherein the biodegradable polymers comprise poly amino acids.

7. The hydrogel of claim 6, wherein the poly amino acids comprise poly glutamic acid (PGA).

8. The hydrogel of claim 1, wherein the biodegradable polymers comprise one or more polysaccharides.

9. The hydrogel of claim 8, wherein the polysaccharides are selected from the group consisting of starch, glycogen, galactogen, arabinoxylans, cellulose, chitin, chitosan, pectin, starch, alginic acid, or combinations thereof.

10. The hydrogel of claim 8, wherein the polysaccharides comprise alginic acid.

11. The hydrogel of claim 1, wherein the biodegradable polymers comprise alginic acid, wherein the galactomannans comprise guar gum, and wherein the alginic acid and the guar gum are covalently cross-linked to one another.

12. The hydrogel of claim 1, wherein the hydrogel is biodegradable.

13. The hydrogel of claim 1, wherein the hydrogel is operable to retain liquids at more than 100 times the hydrogel's weight.

14. The hydrogel of claim 1, wherein the hydrogel is operable to retain liquids at more than 500 times the hydrogel's weight.

15. The hydrogel of claim 1, wherein the hydrogel is a component of an absorptive hygiene product (AHP), wherein the AHP is selected from the group consisting of diapers, tampons, sanitary pads, or combinations thereof.

16. An absorptive hygiene product comprising a hydrogel, wherein the hydrogel comprises:
one or more galactomannans; and
one or more biodegradable polymers.

17. The absorptive hygiene product of claim 16, wherein the one or more galactomannans and the one or more biodegradable polymers are covalently cross-linked to one another.

18. The absorptive hygiene product of claim 16, wherein the galactomannans are selected from the group consisting of natural gums, guar gum, fenugreek gum, xanthan gum, locust bean gum, Arabic gum, tara gum, tragacanth gum, cassia gum, gum karaya, gum ghatti, gellan gum, kondagogu gum, gum tamarind, or combinations thereof.

19. The absorptive hygiene product of claim 16, wherein the galactomannans comprise guar gum.

20. The absorptive hygiene product of claim 16, wherein the biodegradable polymers are selected from the group consisting of agro-polymers, polysaccharides, proteins, poly amino acids, biopolymers, bio-polyesters, or combinations thereof.

21. The absorptive hygiene product of claim 16, wherein the biodegradable polymers comprise one or more polysaccharides.

22. The absorptive hygiene product of claim 21, wherein the polysaccharides are selected from the group consisting of starch, glycogen, galactogen, arabinoxylans, cellulose, chitin, chitosan, pectin, starch, alginic acid, or combinations thereof.

23. The absorptive hygiene product of claim 21, wherein the polysaccharides comprise alginic acid.

24. The absorptive hygiene product of claim 16, wherein the biodegradable polymers comprise alginic acid, wherein the galactomannans comprise guar gum, and wherein the alginic acid and the guar gum are covalently cross-linked to one another.

25. The absorptive hygiene product of claim 16, wherein the absorptive hygiene product is selected from the group consisting of diapers, tampons, sanitary pads, or combinations thereof.

26. The absorptive hygiene product of claim 16, wherein the absorptive hygiene product comprises diapers.

27. A method of making a hydrogel, said method comprising:
associating one or more galactomannans with one or more biodegradable polymers.

28. The method of claim 27, wherein the associating comprises mixing the galactomannans with the biodegradable polymers to form a mixture.

29. The method of claim 27, wherein the associating comprises cross-linking the galactomannans with the biodegradable polymers.

30. The method of claim 27, further comprising a step of incorporating the hydrogel as a component of an absorptive hygiene product.

31. The method of claim 27, wherein the galactomannans are selected from the group consisting of natural gums, guar gum, fenugreek gum, xanthan gum, locust bean gum, Arabic gum, tara gum, tragacanth gum, cassia gum, gum karaya, gum ghatti, gellan gum, kondagogu gum, gum tamarind, or combinations thereof.

32. The method of claim 27, wherein the galactomannans comprise guar gum.

33. The method of claim 27, wherein the biodegradable polymers are selected from the group consisting of agro-polymers, polysaccharides, proteins, poly amino acids, biopolymers, bio-polyesters, or combinations thereof.

34. The method of claim 27, wherein the biodegradable polymers comprise alginic acid.
